(19) United States
(12) Patent Application Publication
(10) Pub. No.: US 2007/0221211 A1
(43) Pub. Date: Sep. 27, 2007

(54) Apparatus and process for inhaling medicines

(76) Inventor: Boris Sagalovich, New York, NY (US)

Correspondence Address: LAW FIRM OF YURI KATESHOV, 174 FERNDALE ROAD, SCARSDALE, NY, 10583, US

(21) Appl. No.: 11/389,861

(22) Filed: Mar. 27, 2006

(51) Int. Cl. A61M 11/00

(52) U.S. Cl. 128/200.15; 128/200.23

(57) ABSTRACT

A tongue protector coupleable to an inhaler is configured with a grip and an elongated body configured to cover at least a portion of the patient's tongue administering a medicine.

### FIELD OF THE INVENTION

The invention relates to inhalers. Particularly, the invention relates to an inhaling assembly provided with a detachable accessory which is operable to protect the patient's tongue during administering a medicine.

### BACKGROUND OF THE INVENTION

Devices operable to allow the patient to inhale pharmaceutical drugs are known in the art. These pharmaceutical drugs are generally introduced into the respiratory tracts via the mouth as an aerosol or powder inhalant by means of a nebulizer or atomizer. In operation, a particular volume of air is enriched with aerosol or powder inhalant in a succession of retarding chambers or spacers before a resulting mixture is being inhaled. These easily portable, non-electrically operated inhalation systems that can be used by the patient himself improve the effectiveness of the inhaled substance.

Quite often, however, a substance to be inhaled may produce undesirable effects on the patient. Varying from mildly unpleasant to very strong reactions by the patient to the inhaled drugs, these effects may compel the patient to stop administering a medicine. For example, dryness of the mouth is the most common side effect of tiotropium bromide (Spiriva®)—an inhalation powder that opens narrowed airways and helps keep them open for many hours. The Spiriva® is administered by a HandiHaler device 10 manufactured by Boehringer Ingelheim Pharma GmbH and is shown in FIGS. 1A and 1B. Generally, device 10 is configured with a dust cup 12 and a mouthpiece 14 each mounted to selectively pivot relative to a base 16 between closed and open positions. In use, a capsule containing the drug is inserted in a chamber 18 provided in base 16, pierced by a piercing button 20, and upon displacing mouthpiece 14 to the closing position as shown by an arrow A (FIG. 1A), the drug is inhaled by the patient through by the tip of mouthpiece 12, as illustrated by an arrow B (FIG. 1B). The mouthpiece 14 is a relatively short, frustoconically shaped component that is typically held by the lips (FIG. 1B) during inhalation. When the powder is inhaled, a portion thereof is deposited upon the tongue.

According to a One-Year-COPD Clinical Trial, up to 16% of patients experience an adverse reaction associated with dry mouth. Furthermore, some clinical observations have shown that protecting the tongue from exposure to Spiriva® substantially minimizes the mouth dryness.

A need, therefore, exists for an accessory for at least some of the known inhalers that would protect the tongue from being exposed to an inhaled medicine.

Another need exists for an accessory for the known inhalers that can be used with these inhalers without modifying the original structure of the inhalers.

Another need exists for an accessory for the known inhalers that has an adjustable structure operable to accommodate a variety of shapes and dimensions associated with the known inhalers;

Still another need exists for an accessory that has an adjustable structure operable to accommodate differently dimensioned oral organs.

Yet a further need exists for a method of administering a variety of inhaleable medicines by utilizing the inventive accessory.

### SUMMARY OF THE INVENTION

These and other needs are satisfied by the present invention. The inventive tongue protector includes a grip and an elongated body defining preferably a one-piece molded body that can be coupled to an inhaler. The elongate body is shaped and dimensioned to cover at least a portion of the tongue during administering a medicine.

The grip may have one or more elongated arms extending from the body and configured to flex upon applying an external force. Each arm has a hook-like end portion engaging the bottom of the inhaler upon mounting the tongue protector to the inhaler in the assembled state of the entire assembly. The flexibility of the arms allows the inventive tongue protector to reliably engage variously shaped and dimensioned inhalers without a need for additional structural modifications of the inventive protector. The new inhalers thus can be manufactured as part of a kit also including the inventive protector. Alternatively, the patient or physician may purchase the inventive protector separately from an inhaler and use the purchased protector with variously shaped and dimensioned inhalers.

The body of the inventive tongue protector is configured with a proximal end shaped and dimensioned to receive a mouthpiece of inhaler in the assembled state of the protector. In accordance with one aspect of the invention, the tongue protector includes a plate having an inner surface which lies atop the substantial portion of the tongue during administering a medicine. The proximal end has a channel receiving the mouthpiece of the inhaler so as to be aligned with the discharge opening of the mouthpiece and guide a medicine from the inhaler towards the plate.

In one aspect, the proximal end of the protector and the mouthpiece frictionally engage one another to provide a tight-fit coupling therebetween. Upon coupling, the peripheral surface of the channel conforms to the outer surface of the mouthpiece. In a further aspect of the invention, the peripheral surface of the channel threadedly engages the outer surface of the mouthpiece.

The inventive tongue protector also has a support plate coupled to the protector's body so that the distal portion of the body and the support plate define a space therebetween configured to receive the tongue of the patient. Advantageously, the support plate is slidably mounted to the tongue protector so as to adjust the overall dimensions of the protector in accordance with the individual anatomical particularities of the patient.

In accordance with another embodiment of the invention, the tongue protector includes an elongated hollow body having its inner surface define a channel guiding a medicine past the tongue. The channel is aligned with a discharge opening provided in a mouthpiece of inhaler upon coupling the tongue protector to the body of the inhaler.

The elongated hollow body of the protector may have a one-piece structure. According to another modification, the body is configured with a telescopically coupled components providing for the adjustment of the overall length of the tongue protector in accordance with the patient's anatomical particularities.

A further embodiment of the invention is directed to an inhaler assembly including an inhaling device configured to discharge a medicine through a mouth of a patient, and a tongue protector detachably coupled to the inhaling device and configured to substantially cover a tongue of the patient upon actuating the inhaling device.

In accordance with still a further embodiment of the invention, a method of using an inhaling device configured to substantially minimize dryness of the mouth is disclosed.

The aforementioned features and advantages of the invention will be pointed out with particularity, and will become obvious from the following detailed description of the invention, taken in conjunction with the accompanying drawings, which form an integral part thereof.

### BRIEF DESCRIPTION OF THE DRAWINGS

FIGS. 1A and 1B are views illustrating the known prior art device and their use;

FIG. 2 is an elevated side view of the inventive inhaling assembly;

FIG. 3 is perspective view of the inventive inhaling assembly configured in accordance with one embodiment of the invention;

FIG. 4 is an exploded view of the assembly shown in FIG. 3;

FIG. 5 is a perspective view of the inventive assembly configured in accordance with a further embodiment of the invention; and

FIG. 6 is an exploded view of the assembly of FIG. 5.

### SPECIFIC DESCRIPTION

Reference will now be made in detail to several embodiments of the invention that are illustrated in the accompanying images. The images are in simplified form and are not to precise scale. For purposes of convenience and clarity only, directional terms, such as top, bottom, left, right, up, down, above, below, proximal, and distal may be used with respect to the drawings. These and similar directional terms should not be construed to limit the scope of the invention in any manner. The words “connect,” “couple,” “attach” and similar terms with their inflectional morphemes do not necessarily denote direct and immediate connections, but also include connections through mediate elements or devices. The terms “tongue protector” and “accessory” are used hereinbelow interchangeably.

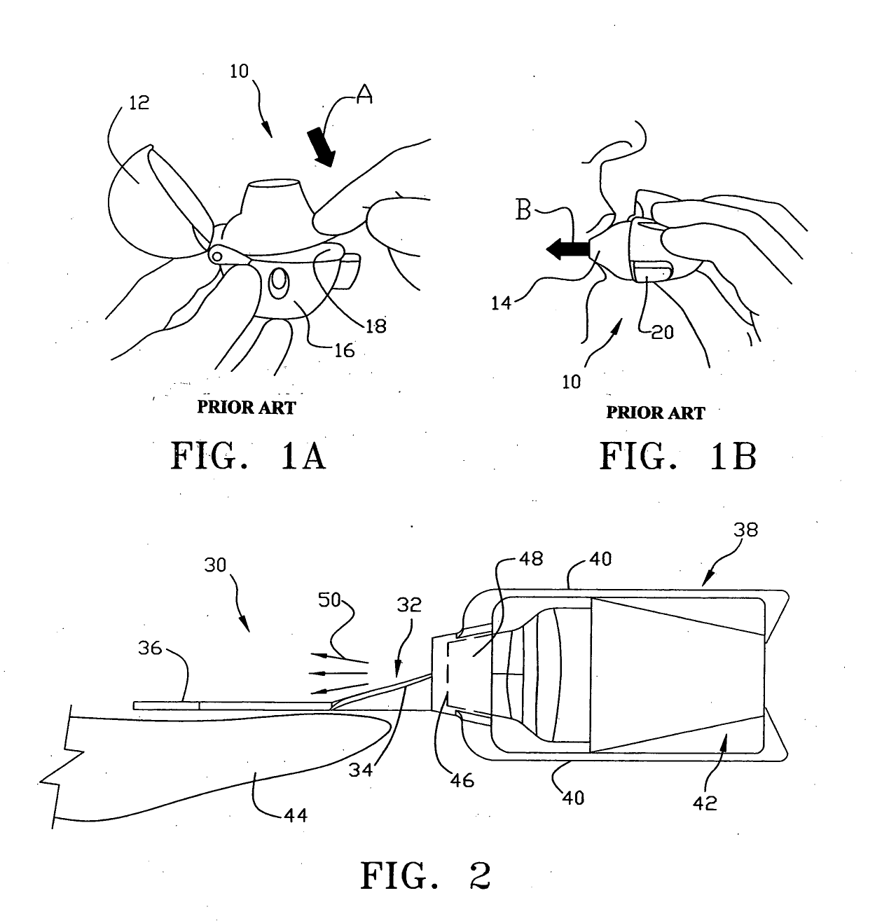

Referring to FIG. 2, the inventive assembly 30 is configured with an accessory 32 coupleable to an inhaler 42 for administering a medicine 50. The accessory 32 is structured to protect the patient's tongue 44 upon actuating inhaler 42 and has a grip 38 adjustable to engage variously shaped and dimensioned inhalers. The inhaler 42 may be configured, for example, as try powder inhaler 10 shown in FIGS. 1A and 1B. As will be disclosed hereinbelow, accessory 32 can be used within variously shaped, dimensioned and operated inhalers including for example metering dosing inhalers.

In use, while the patient is pushing a proximal end 46 of accessory 32 onto a mouthpiece 48 of inhaler 42, a pair of arms 40 of grip 38 slide along the inhaler and grip its bottom preventing, thus, voluntary displacement of accessory 32 and inhaler 42 relative to one another. In the assembled state, a distal portion 36 of accessory 32 lies atop patient's tongue 44 and thus prevents contact between the tongue and medicine 50.

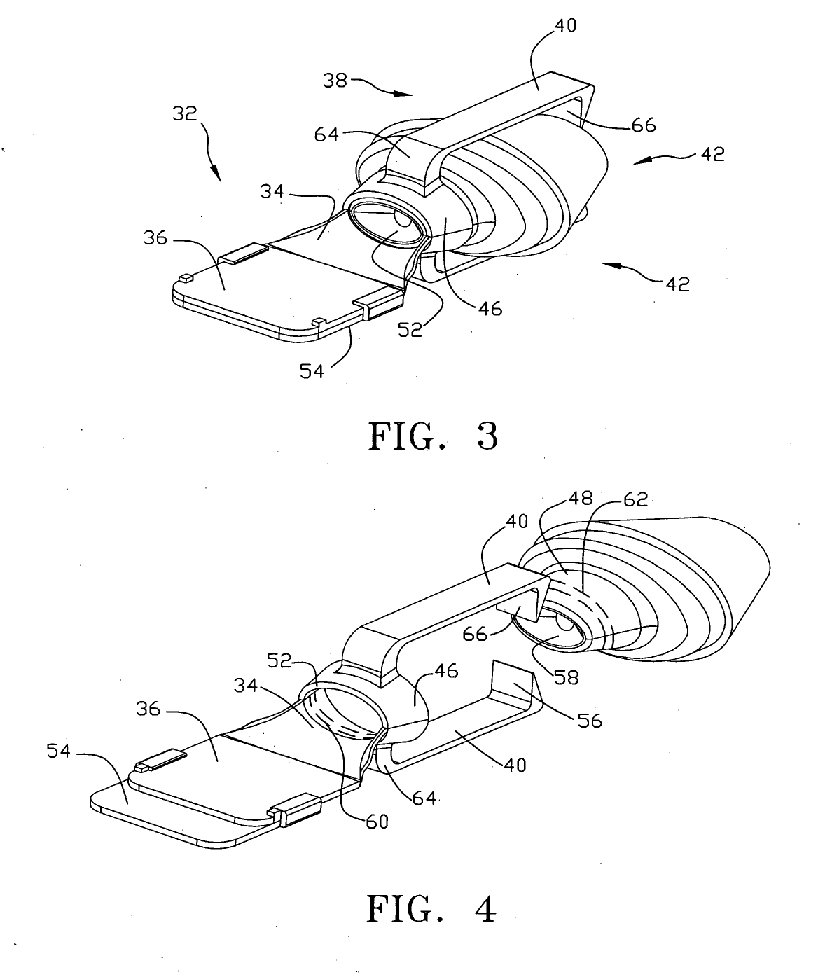

Turning now to FIGS. 3 and 4, accessory 32 includes flat distal portion 36 configured to cover the patient's tongue, a slanted intermediary portion 34 and proximal portion 46 having a frustoconically shaped cross-section. The proximal portion 46 of accessory 32 includes a tubular, frustoconical body having a channel 52 which opens at the opposite ends of proximal portion 46. The channel 52 is configured to receive mouthpiece 48 so as to be aligned with a discharge opening 58 of mouthpiece 48 of the inhaler in the assembled state of inventive assembly 30.

As is also shown in FIGS. 3 and 4, mouthpiece 48 of inhaler 42 is generally frustoconical. To provide a reliable tight engagement between accessory 32 and inhaler 42, the inner surface of proximal portion 46 and the outer surface of mouthpiece 48 frictionally engage and extend complementary to one another in the assembled state of assembly 30, as shown in FIG. 3. Alternatively, the inner and outer surfaces of accessory 32 and inhaler 42, respectively, may have respective threads 60 and 62 engaging one another by rotating the accessory and inhaler relative to one another. An ordinary skilled worker can easily see that although the mating inner and outer surfaces of respective proximate portion 46 and mouthpiece 48 are shown to be generally frustoconical, other cross-sections including, but not limited to, round, oval and polygonal, can be easily implemented within the scope of this invention depending on the configuration of inhaler 42.

The intermediary portion 34 of accessory 32 extends angularly inwards from proximal portion 46 of accessory 32 and allows distal, plate-like portion 36 to press upon the patient's tongue. The distal portion 36 is shaped and dimensioned to extend substantially across the entire patient's tongue and may have rounded edges to prevent damage to the patient.

To maximize comfort of the patient during administering a medicine, distal portion 36 may be alternatively provided with a support plate 54. The distal portion 36 and support plate 54 may be located next to one another, as shown in FIGS. 3 and 4. Optionally, distal portion 36 and plate 54 may define a space therebetween sufficient to receive the patient's tongue (not shown). The support plate 54 may be slidably mounted to the distal portion 34. Displacing support plate 54 relative to distal portion 36 helps the patient adjust the length of the space between these components and accommodate differently dimensioned tongues. The displaceable plate 54 may be optionally mounted on top of distal portion 36.

The arms 40 of accessory's grip 38 each have respective a distal end 64 fixed to proximal portion 46 of accessory 32 and an elongated body extending from the distal end over the entire length of inhaler 42 in the assembled state of assembly 30. The proximal end 66 of arms 40 is substantially hook-shaped to engage the bottom of inhaler 42 in the assembled state of assembly 30. Made from flexible material, the arms easily flex to accommodate any shape and form of inhaler 42. In addition, each of arms 40 may have a couple of arm components telescopically coupled to one another so as to accommodate variously dimensioned inhaling devices and shown only diagrammatically as being well known in the art. Although the telescopic structure of the arms is not shown, the basic principle of operation of such a structure is well known to those of ordinary skill.

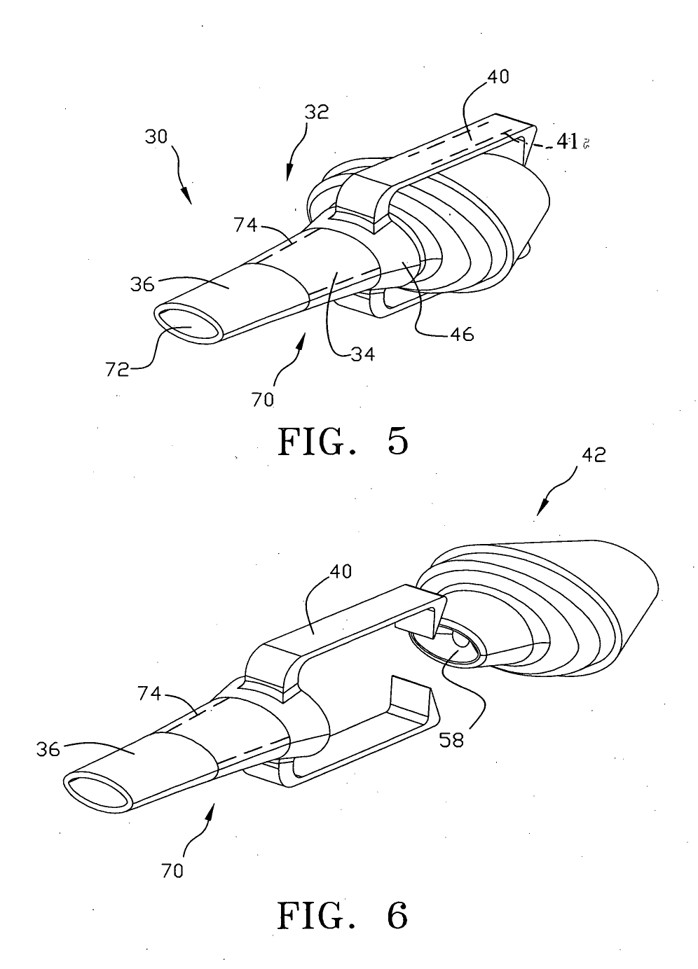

Referring to FIGS. 5 and 6, accessory 32 is configured in accordance with a further embodiment and includes an elongated hollow body 70 provided with conically shaped proximal portion 46, intermediary portion 34 and distal portion 36. The body 70 is configured to extend desirably along the entire length of the patient's mouth so as to be able to protect the patient's tongue from contact with a medicine.

The proximal portion 46 is configured to receive the mouthpiece of inhaler 42 and is displaceably fixed thereto upon engaging between the free ends of arms 40 with the bottom of inhaler 42 in the assembled state of assembly 30. The assembled state of assembly 30 is characterized by alignment between an inner channel 72 of hollow body 70 and discharge opening 58 of inhaler 42. Coupling between accessory 32 and inhaler 42 is similar to the embodiment disclosed in reference to FIGS. 2 and 3 and includes a tight fit or threaded connection.

The body 70 may be molded in one piece. Alternatively, intermediary portion 34 and distal portion 36 of body 70 may be telescopically coupled to one another, as diagrammatically indicated by a numeral reference 74. The patient may, thus, adjust the length of body 70 according his/her anatomical particularities. Optionally, arms 40 each may have a telescopic structure 41, as diagrammatically shown in phantom lines in FIG. 5, and engage inhalers 42 having various shapes and dimensions.

The accessory 30 may be manufactured separately from inhaler 42. Alternatively, a kit including inhaler 42 and accessory 32 can be manufactured. The patient may use up the medicine, including powder or aerosol, contained in inhaler 42 and purchase another inhaler that can be further used with the previously purchased accessory in accordance with the disclosed method of use.

The specific features described herein may be used in some embodiments, but not in others, without departure from the spirit and scope of the invention as set forth. Many additional modifications are intended in the foregoing disclosure, and it will be appreciated by those of ordinary skill in the art that in some instances some features of the invention will be employed in the absence of a corresponding use of other features. The illustrative examples therefore do not define the metes and bounds of the invention.

1. A tongue protector coupleable to an inhaler, comprising:
a grip, and
an elongated body configured to cover at least a portion of a tongue during administering a medicine.

2. The tongue protector of claim 1, wherein the grip has at least one elongated arm extending from the body and configured to flex upon applying an external force, the at least one arm having a free end operable to engage the inhaler.

3. The tongue protector of claim 2, wherein the free end of the at least one arm is configured with a hook extending transversely to the arm.

4. The tongue protector of claim 3, further comprising an additional elongated arm spaced angularly from the one elongated arm, the at least one and additional elongated arms being operable to deflect from one another in opposite directions in response to applying the external force and each having a telescopic structure for adjusting a length of the arm.

5. The tongue protector of claim 1, wherein the elongated body has proximal and distal portions and is shaped and dimensioned to cover at least a substantial portion of a tongue.

6. The tongue protector of claim 5, wherein the proximal end is configured to receive a discharge end of the inhaler upon coupling the tongue protector to the inhaler, the proximal end having a channel aligned with an opening, which is provided in the discharge end of the inhaler, upon coupling the tongue protector to the inhaler.

7. The tongue protector of claim 6, wherein the channel has an inner surface shaped and dimensioned to frictionally engage an outer surface of the discharge end of the inhaler.

8. The tongue protector of claim 6, wherein the channel has an inner surface threadedly engaging an outer surface of the discharge end of the inhaler.

9. The tongue protector of claim 5, wherein the body further comprises an intermediary portion extending between the proximal and distal portions, the intermediary portion angularly extending from the proximal portion and being configured to allow uninhibited flow of the medicine upon coupling the tongue protector to the inhaler.

10. The tongue protector of claim 9, wherein the intermediary and distal portions of the body are flat.

11. The tongue protector of claim 10, further comprising a support plate coupled to the body so that the distal portion of the body and the support plate define a space therebetween configured to receive a tongue.

12. The tongue protector of claim 11, wherein the support plate is displaceably mounted to the distal portion of the body.

13. The tongue protector of claim 5, wherein the body has an annularly shaped cross-section defining an inner channel for guiding a medicine upon coupling the tongue protector to the inhaler.

14. The tongue protector of claim 13, wherein the proximal and distal portions of the body are telescopically coupled to one another.

15. An inhaler assembly, comprising:
an inhaling device configured to discharge a medicine through a mouth of a patient; and
a tongue protector detachably coupled to the inhaling device and configured to substantially cover a tongue of the patient upon actuating the inhaling device.

17. The inhaler assembly of claim 16, wherein the inhaling has a mouthpiece provided with a discharge opening, the tongue protector comprising:
a body configured to allow an uninhibited flow of a medicine, and
a pair of spaced apart flexible arms coupled to the body and operable to extend along and releasably engage the inhaling device.

17. The inhaler assembly of claim 16, wherein the mouthpiece of the inhaling device has one of a polygonally-shaped cross-section, circularly-shaped cross-section and irregularly-shaped cross-section, the body of the tongue protector having an inner surface defining a channel and extending complementary to an outer surface of the mouthpiece so that a discharge opening of the mouthpiece and the channel are aligned upon coupling the tongue protector and the inhaling device.

18. The inhaler assembly of claim 16, wherein the tongue protector is configured with a plate lying atop the tongue of the patient and substantially covering an entire surface thereof.

19. The inhaler assembly of claim 16, wherein the body of the tongue protector has an inner channel extending through an entire length of the body, the arms each having a telescopic structure so as to allow the arms to accommodate variously dimensioned inhalers.

20. A method of administering a medicine, comprising detachably coupling a tongue protector to an inhaling device so as to substantially cover a tongue of a patient during administering the medicine.
